(19) United States
(12) Patent Application Publication
(10) Pub. No.: US 2005/0157260 A1
(43) Pub. Date: Jul. 21, 2005

(54) Lens for producing stereoscopic images

(76) Inventors: Raymond D. Graham, Renton, WA (US); Peter G. Harrington, Seattle, WA (US); Giovanni Staurenghi, Milan (IT)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/987,204

(22) Filed: Nov. 12, 2004

Related U.S. Application Data
(60) Provisional application No. 60/519,776, filed on Nov. 12, 2003.

(51) Int. Cl. A61B 3/00; A61B 3/14

(52) U.S. Cl. 351/219

(57) ABSTRACT

A stereoscopic imaging lens includes a first lens set and a second lens set. The lens receives collimated laser light from the scanning laser ophthalmoscope and, focuses on the fundus of the eye. By use of a prism set, two offset images are provided to the scanning laser ophthalmoscope. The system provides virtually simulatneous side-by-side but offset images that can be viewed with a stereoscopic viewing device, which provides apparent depth perception.

### CROSS-REFERENCE TO RELATED APPLICATION

This application claims the benefit of Provisional Application No. 60/519,776, filed Nov. 12, 2003, the benefit of which is hereby claimed under 35 U.S.C. § 119.

### FIELD OF THE INVENTION

The present invention relates to a lens system for use with ophthalmoscopes and particularly scanning laser ophthalmoscopes, to provide substantially simultaneous side-by-side views of a selected region of the eye for use in producing a stereoscopic image.

### BACKGROUND OF THE INVENTION

Scanning laser ophthalmoscopes are capable of providing high quality video images of the retina using lower light levels than those required for conventional fundus photography or indirect ophthalmoscopy. In a scanning laser ophthalmoscope (SLO), a low power laser beam is employed to scan across the retina. The reflected light is then gathered by the SLO and converted into a video image. The instrument is highly light efficient, using illumination levels that are comfortable and safe for the patient. In addition, the scanning laser ophthalmoscope can be used for retinal angiography and autofluorescence imaging.

The SLO is also utilized to produce stereoscopic images. This is accomplished with the standard scanning laser ophthalmoscope by taking a first image of the eye and then adjusting the angle of the ophthalmoscope relative to the eye by one to three degrees to produce a second image that is slightly offset from the first. The side-by-side images are then displayed on the SLO's video monitor associated with the ophthalmoscope. Side-by-side images are then viewed with conventional stereoscopic viewing devices that create for the observer an apparent depth in the images that can aid in diagnosing eye conditions. The disadvantage of this method is that the orientation of the SLO relative to the patient's eye must be changed to produce the stereo pair of images. This manual change in SLO orientation induces a time difference between images. Furthermore, orientation may differ from one stereoscopic image pair to the next introducing differences in the stereoscopic depth of sequential images.

### SUMMARY OF THE INVENTION

Our invention provides a stereoscopic image pair of fixed stereo depth without manually changing the orientation of the SLO relative to the patient's eye. The present invention therefore provides a lens that can be positioned between the eye and a standard scanning laser ophthalmoscope that with a scan from a single position simultaneously produces paired images that can then be used for stereoscopic viewing. The lens comprises first and second sets of optical elements. In a preferred embodiment, the first set of optical elements includes a first lens having a posterior surface that can contact the eye and a convex anterior surface. The second lens of the first set is preferably a bi-convex aspheric lens. The combination of these two lenses forms an aerial image anterior to the anterior surface of the second lens element. The second set of optical elements is positioned anterior to the aerial image. The first element comprises a bi-convex aspheric lens that takes collimated light from the scanning laser ophthalmoscope and in combination with the first pair of elements focuses it on a predetermined position on the retina of the eye. When the reflected light rays from the image travel outwardly from the eye, they first form an aerial image anterior to the first pair of elements and then are collimated by the aspheric lens of the second set of optical elements. The second set of elements also includes a pair of prisms that are positioned anterior to the first element of the second set. The prisms meet in an apex which in plain view intersects and is perpendicular to the optical axis of the total lens system. When the light from the scanning laser ophthalmoscope scans from one side of the optical axis, the rays are refracted by the prism so that they are focused on a predetermined location on the retina. As the scanning laser ophthalmoscope scans from the opposite side of the optical axis, the laser focuses on a second region of the retina, slightly offset from the first region of the retina. When these light rays are reflected back out to the scanning laser ophthalmoscope, two side-by-side images are produced. These images are time differentiated only by the period of the scan of the scanning laser ophthalmoscope, which is on the order of 48-96 milliseconds. Thus, two side-by-side, slightly offset images are produced by the ophthalmoscope that can be viewed by a stereoscopic viewer that eliminates the drawbacks of the prior methods.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a longitudinal cross section of the lens of the present invention taken along a line intersecting the optical axis of the lens;

FIG. 2 is a plain view of the anterior portion of the lens taken along view line 2-2 of FIG. 1;

FIGS. 3A and 3B are diagrammatic views showing ray tracings of the light emanating from and reflected back to the scanning laser ophthalmoscope using the lens shown in FIGS. 1 and 2;

FIG. 4A is a schematic diagram of the scanning laser ophthalmoscope used to produce an image in accordance with the present invention;

FIG. 4B is a representation of an image produced in accordance with the present invention;

FIGS. 5A and 5B are diagrammatic views showing ray tracings of light emanating from and being reflected back to a scanning laser ophthalmoscope using a non-contact lens embodiment of the present invention; and

FIG. 6 is a diagrammatic view of another embodiment of the invention similar to that shown in FIGS. 5A and 5B.

### DETAILED DESCRIPTION OF THE PREFERRED EMBODIMENT

In its preferred form, the stereoscopic imaging lens 10 is housed in a conical housing 12 holding a first set of lens elements 14 and a second set of lens elements 16. The first and second sets of elements 14 and 16 are aligned by the housing 12 along an optical axis 18 upon which each of the lenses of the elements is centered. The first set of optical elements 14 includes a contact lens 20 and a bi-convex aspheric lens 22. The contact lens 20 has a concave posterior surface 24 that has a curvature corresponding to the average curvature of the human cornea. The anterior surface 26 of the contact lens 20 is convex and in the preferred embodiment, spherical. The aspheric lens 22 has a posterior convex aspheric surface 28 and an anterior convex aspheric surface 30.

In use, the posterior surface 24 of the contact lens 20 is positioned against the cornea of the eye with a suitable optical coupling fluid such as methylcellulose. The first set of elements 14 serves to take light rays reflected from the retina of the eye and focus them in an aerial image plane 32 positioned anterior from the aspheric lens 22. Light rays extending anteriorly from the aerial image 32 then traverse the second lens of the set of lens elements 16.

The second set of lens elements 16 comprises a bi-convex aspheric lens 40 having a convex posterior surface 42 and a convex anterior surface 44. The second set of optical elements 16 further comprises a pair of prisms 46A and 46B. The apexes of the prisms 46A and 46B meet along a straight line 48 that intersects and is perpendicular to the optical axis 18 of the lens system. The upper surfaces of each of the prisms 46A and 46B are planar and extend posteriorly and laterally outwardly, respectively, from the line 48. The outer edges of the prisms 46A and 46B are circular so as to conform to the shape of the housing 12. The posterior surfaces 50A and 50B of the prisms 46A and 46B are coplanar and are aligned perpendicularly to the optical axis 18. The posterior surfaces 50A and 50B are positioned anteriorly from the anterior surface 44 of the aspheric lens 40.

In use, the aspheric lens 40 receives the light rays from the aerial image 32 and collimates them so that when they pass through the prisms 46A and 46B they are parallel. In reverse, the scanning laser light from the scanning laser ophthalmoscope is refracted by the prisms 46A and 46B, is focused by the aspheric lens on the plane of the aerial image 32, and is refocused by the first lens set 14 to provide illumination for the retina of the eye.

In the preferred embodiment, the posterior and anterior contact lens surfaces 24 and 26 are spherical and have radii of curvature of 7.4 mm and 9 mm, respectively. The thickness of the contact lens along the optical axis is 5.5 mm. The contact lens is preferably composed of polymethylmethacrylate. The air gap between the contact lens and the surface 28 of the aspheric lens 22 is about 0.5 mm.

The curvatures of the aspheric surfaces are determined by the formula:

$$z = \frac{{CK}^{2}}{1 + \sqrt{1 - {C}^{2} {EK}^{2}}} , \\ wherein C = \frac{1}{R} , \\ E = b + 1 , and$$

$${K}^{2} = {x}^{2} + {y}^{2} ,$$

wherein z is the axial position of the curved surface,
x and y are the coordinates perpendicular to the optical axis,
R is the radius of curvature, and
b is the conic constant.

For the posterior surface of lens 22, R is 14.5 mm and b is −1.5. For the anterior surface of lens 22, R is 23.5 and b is −4.3. The thickness of lens 22 along the optical axis is 7.7 mm. For the posterior surface of lens 40, R is 35.0 mm and b is −7.6. For the anterior surface of lens 40, R is 18.0 and b is −1.7. The thickness of lens 40 along the optical axis is 12.1 mm. The air gap between lenses 22 and 40 is 21.3 mm, and between the anterior surface of lens 40 and the posterior surfaces of prisms 46A, 46B, the air gap is 1.0 mm. Lenses 22 and 40 and the prisms are made from optical glass. Optionally, they may be made from polymeric material such as polymethylmethacrylate. The anterior surfaces of prisms 46A, 46B are inclined at 17° from a plane perpendicular to the optical axis.

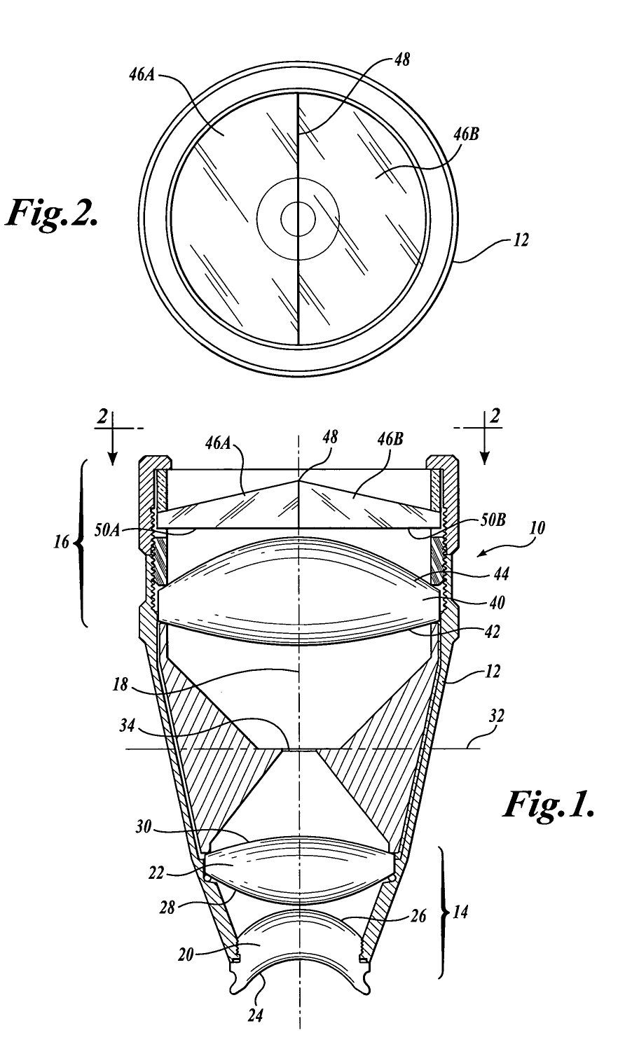

It is also preferred that a field stop be employed between the two lens sets, and preferably at the aerial image plane 32. As shown in FIG. 1, this field stop 34 has a diameter of 3.8 mm and is centered on the optical axis 18.

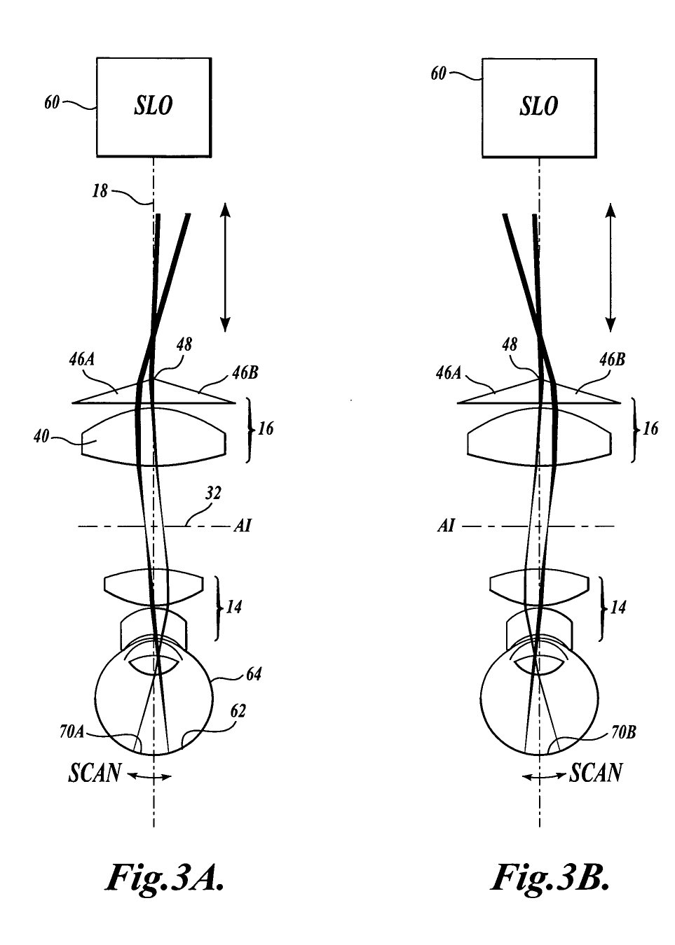

Referring now to FIGS. 3A and 3B, a set of ray tracings shows laser light emanating from a scanning laser ophthalmoscope 60 traversing the second lens set 16 and the first lens set 14 and focused on the fundus 62 of an eye 64. FIG. 3A shows the rays as they are traced from the right side of the optical axis 18 through the left prism 46A. The rays are shown collimated passing through the prisms 46A and 46B. They are focused by the aspheric lens 40 on the image plane 32 and refocused by the second set of lens elements 16 on the fundus 62. This laser light is then reflected by the fundus in a reverse direction through the first and second lens sets 14 and 16 and back to the scanning laser ophthalmoscope 60 which converts the reflected rays into an image. As seen in FIG. 3A, the rays from the right side of the optical axis 18 that pass through the axis anterior to the prisms 46A, 46B and intersect the left prism 46A, scan an image area on the fundus 62 which is slightly offset to the left of the optical axis 18. Referring to FIG. 3B, the rays, when scanning from the left side of the optical axis 18, passing through the axis anterior to prisms 46A, 46B, and intersecting the right prism 46B, scan an area on the fundus 62 that is slightly offset to the right of the optical axis 18. As the scanning rays pass over the line 48 of the prism, the image is immediately shifted from left to right so that the image formed by the rays passing through prism 46A are slightly offset from the image formed by the rays passing through prism 46B.

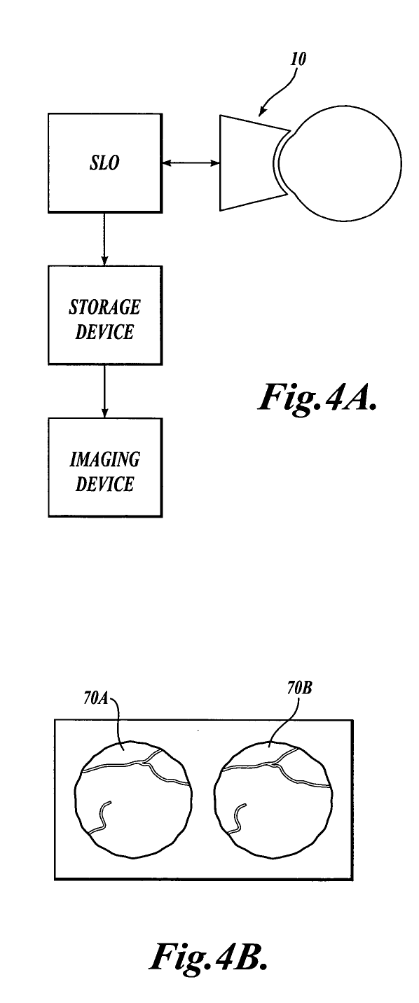

Referring to FIG. 4A, the scanning laser ophthalmoscope 60 includes a storage device for storing images and an imaging device such as an LCD or CRT display. The lens 10 of the present invention produces two images 70A and 70B from the fundus 62 of the eye (FIG. 4A) corresponding to the image areas 70A and 70B prescribed on the fundus in FIGS. 3A and 3B. As can be seen in FIG. 4B, these images are slightly offset from each other. When these images, shown in FIG. 4B, are viewed stereoscopically the viewer is able to perceive depth in the image and thus can see features of the retina that are not otherwise discernable in the separate images. As mentioned above, the two images 70A and 70B have virtually no time delay between them except for the time delay of a single scan of the laser ophthalmoscope.

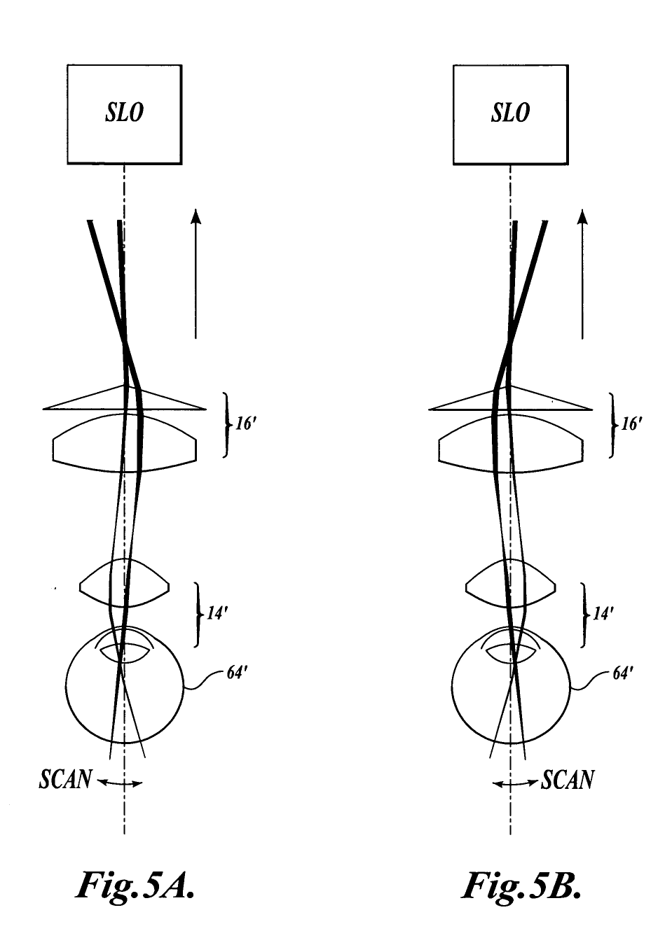

Referring now to FIGS. 5A and 5B, a second embodiment of the first and second lens sets, 14′ and 16′, is illustrated. In this embodiment the lens set 14′ comprises a single bi-convex aspheric lens which is spaced from the cornea of the eye 64′. This embodiment is useful because it does not require topical anesthetic. It also simplifies the physician's or technician's tasks by eliminating the need for a contact lens to be manually held on the patient's eye. The lens set shown in FIGS. 5A and 5B otherwise functions virtually identically to the lens set shown and described in the previous Figures.

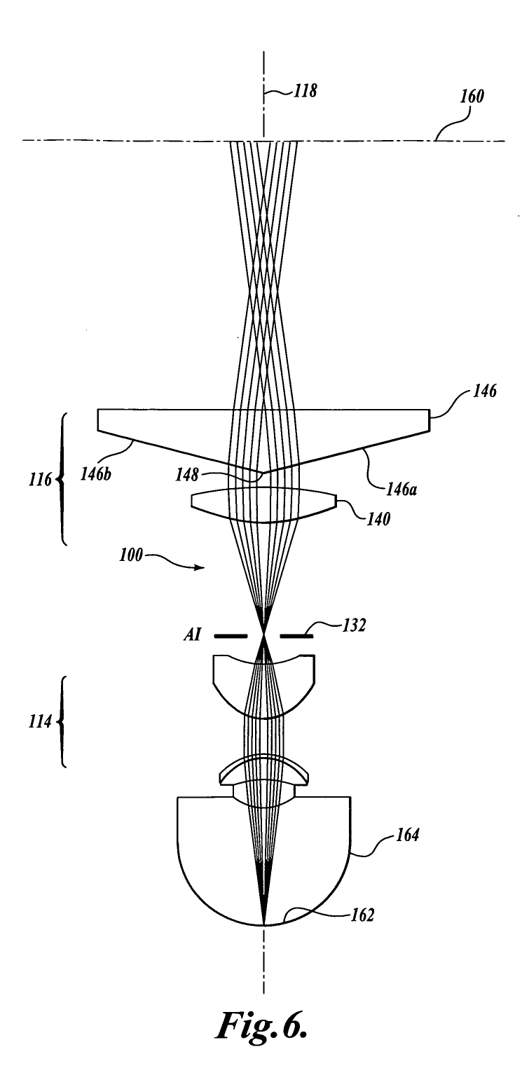

Referring now to FIG. 6, another embodiment of the invention similar to that shown in FIGS. 5A and 5B is diagramatically illustrated. In this embodiment, the lens 100 comprises first to lens set 114 and second lens set 116 corresponding to lens sets 14′ and 16′ of the FIGS. 5A and 5B. In this embodiment, the first lens set comprises a single aspheric lens with a concave anterior surface and a convex posterior surface. This lens focuses light rays reflected from the retina 162 of the eye 164 at an aerial image plane AI anterior to the eye.

In this embodiment a field stop 132 is positioned at the location of the aerial image plane AI formed by lens set 114. The field stop may be circular or rectangular in configuration depending upon the desired end images. The field stop may be utilized on all of the embodiments herein. For example, a circular field stop was utilized to produce the circular images schematically shown in FIG. 4B.

The lens set 116 is similar to the lens set 16′ of FIGS. 5A and 5B with the exception that the prism 146 is inverted with the apex 148 lying on the optical axis 118 and perpendicular thereto. The apex 148 is thus juxtaposed adjacent the anterior convex surface of the aspheric lens 140. In this embodiment, the angular planar surfaces 146A and 146B intersect the optical axis 118 at an angle on the order of 14.47°. This configuration allows further separation of the two images produced by the lens system. Increased separation of the two images produces better three-dimensional depth when viewed stereoscopically.

While the preferred embodiment of the invention has been illustrated and described, it will be appreciated that various changes can be made therein without departing from the spirit and scope of the invention. For example, the lens can be used with any of a variety of conventional illumination sources and imaging devices. Examples of imaging devices include conventional and digital cameras, fundus cameras and the like. Examples of illumination sources include handheld and mounted lights, illuminated microscopes, slit lamps and the like.

1. An ophthalmic lens system usable with a scanning laser ophthalmoscope (SLO) comprising:
a first lens system aligned on an optical axis positioned anterior to the eye, the first lens system capable of focusing light reflected from the retina of the eye in an image plane anterior to the eye; and
a second lens system aligned on the optical axis including a first element capable of receiving light rays from the image plane and collimating and directing those light rays to a SLO, and for receiving collimated light from said SLO and focusing said light on said image plane for redirection through said first lens system to focus on the retina of the eye, said second lens system including a second element for directing light entering said second lens system from one side of said optical axis to a predetermined location on said retina, and for directing light entering said second lens system from the other side of said optical axis to a second predetermined location on the retina offset from the first predetermined location on the retina, the light reflected from the first and second predetermined locations being directed by the first and second lens system to said SLO.

2. The lens of claim 1, wherein said side-by-side images are viewable on said SLO by a stereoscopic viewer to enable depth perception.

3. The lens of claim 1, wherein said first lens system comprises a contact lens and a second lens for focusing light from said retina on said image plane.

4. The lens of claim 3, wherein the second element of said second lens system is a prism having an apex residing on a line intersecting and perpendicular to the optical axis.

5. The lens of claim 4, wherein said prism has first and second elements having individual apexes that meet at said line.

6. The lens of claim 4, wherein said apex is anterior relative to said second element.

7. The lens of claim 4, wherein said apex is posterior relative to said second element.

8. The lens of claim 4, wherein said first element of said second lens system is a bi-convex aspheric lens.

9. The lens of claim 8, wherein the second lens of said first lens system is a bi-convex aspheric lens.

10. The lens of claim 1, wherein said first lens system comprises only an aspheric lens.
